(19) United States
(12) Patent Application Publication
(10) Pub. No.: US 2026/0272319 A1
(43) Pub. Date: Sep. 17, 2026

(54) MODULAR USER HEALTH STATUS MONITORING DEVICE, METHOD, AND SYSTEM

(71) Applicant: HANGZHOU MEGASENS TECHNOLOGY CO., LTD., Hangzhou (CN)

(72) Inventors: Congcong ZHOU, Hangzhou (CN); JianJun Wang, Hangzhou (CN); Jun Hu, Hangzhou (CN); Xu Fan, Hangzhou (CN); Haiquan Yuan, Hangzhou (CN)

(21) Appl. No.: 19/670,166

(22) Filed: May 7, 2026

Related U.S. Application Data
(63) Continuation of application No. PCT/CN2024/131968, filed on Nov. 14, 2024.

(30) Foreign Application Priority Data
Apr. 8, 2024 (CN) 202410417852.4

(51) Int. Cl. A61B 5/0295; A61B 5/00; A61B 5/01; A61B 5/145; A61B 5/1455

(52) U.S. Cl. CPC A61B 5/0295; A61B 5/01; A61B 5/14532; A61B 5/14552; A61B 5/6824; A61B 5/6826; A61B 5/7235; A61B 5/7271; A61B 5/742; A61B 2560/0204; A61B 2560/0443; A61B 2560/0468; A61B 2562/028

(57) ABSTRACT

Provided are a modular user health status monitoring device, method, and system, relating to the technical field of health status monitoring. The device includes: a plurality of functional components, where the plurality of functional components are detachably connected end to end to form a wristband or a ring; a sensing detection module, located in any one of the functional components; and a main control module, located in any one of the functional components and connected to the sensing detection module. The modules in this application can be easily detached and arbitrarily combined, enabling convenient implementation of different measurement functions.

### CROSS REFERENCE TO RELATED APPLICATION

This application is a continuation of International Patent Application No. PCT/CN2024/131968, filed on Nov. 14, 2024, which claims priority to the Chinese Patent Application No. 202410417852.4, filed with the China National Intellectual Property Administration (CNIPA) on Apr. 8, 2024, and entitled “MODULAR USER HEALTH STATUS MONITORING DEVICE, METHOD, AND SYSTEM”, which is incorporated herein by reference in its entirety.

### TECHNICAL FIELD

The present disclosure relates to the technical field of health status monitoring, and in particular, to a modular user health status monitoring device, method, and system.

### BACKGROUND

Currently, various types of smart wearable devices have emerged, including those for human health, virtual reality and augmented reality, and information services. Smart wearable devices are constantly evolving to be more practical, intelligent, and convenient, bringing convenience to people's lives. However, the accuracy of monitoring data from current wearable devices needs further improvement. For different application scenarios, a wide variety of wearable devices are usually required to meet different application needs. In the future, wearable devices will adapt to more application scenarios, including mobile healthcare and the broader health sector. Aspects such as user experience, battery life, and device size will be continuously optimized.

Therefore, how to provide a user health status monitoring device where each module is detachable and can be arbitrarily combined to achieve different measurement functions has become an urgent problem to be solved in the field.

### SUMMARY

An objective of the present disclosure is to provide a modular user health status monitoring device, method, and system, where each module can be easily detached and arbitrarily combined, enabling convenient implementation of different measurement functions.

To achieve the above objective, the present disclosure provides the following technical solutions.

According to a first aspect, the present disclosure provides a modular user health status monitoring device, including:
a plurality of functional components, wherein the plurality of functional components are detachably connected end to end to form a wristband or a ring;
a sensing detection module, located in any one of the functional components; and
a main control module, located in any one of the functional components and connected to the sensing detection module.

In one embodiment, the monitoring device further includes: a storage module, located in any one of the functional components and connected to the main control module.

In one embodiment, the monitoring device further includes: a power module, located in any one of the functional components and connected to the sensing detection module and the main control module.

In one embodiment, the monitoring device further includes: a display module, located in any one of the functional components and connected to the main control module.

In one embodiment, the monitoring device further includes: a data communication module, located in any one of the functional components and connected to the main control module.

In one embodiment, the sensing detection module includes: a Micro-Electro-Mechanical System (MEMS) gyroscope, an acceleration sensor, a heat flux sensor, a thermal resistance sensor, an electrochemical/fluorescence reaction sensor, a high-frequency electrical excitation electrode, a weak electrical detection electrode, an ink printing sensor, a photoelectric sensor, and a laser sensor.

According to a second aspect, based on the aforementioned device of the present disclosure, the present disclosure further provides a modular user health status monitoring method, where the monitoring method is applied to the device according to the first aspect, and the monitoring method includes: acquiring a photoplethysmography (PPG) signal; calculating blood perfusion based on the PPG signal by using a decision algorithm; acquiring a surface temperature of a local tissue; and calculating a core temperature of a to-be-measured site based on the blood perfusion and the surface temperature of the local tissue.

In one embodiment, the monitoring method further includes: calculating blood glucose of a user, specifically including:
collecting blood glucose monitoring data, where the blood glucose monitoring data includes: timestamps and corresponding blood glucose, as well as multi-wavelength PPG original signals, core temperature Tc of the to-be-measured site, and peripheral capillary oxygen saturation (Spo2) of the to-be-measured site;
preprocessing the blood glucose monitoring data; constructing a plurality of deep learning sub-models; training the plurality of deep learning sub-models based on the preprocessed blood glucose monitoring data; performing weighted fusion on the plurality of trained deep learning sub-models using a model fusion algorithm to obtain a final blood glucose prediction model; and predicting the blood glucose of the user based on the blood glucose prediction model.

In one embodiment, a formula for calculating the core temperature of the to-be-measured site based on the blood perfusion and the surface temperature of the local tissue is as follows:

$${T}_{c} = {T}_{0} + \frac{{q}_{1}}{k \sqrt{λ * ω}} + {k}_{1} R ;$$

where T_{c} represents the core temperature of the to-be-measured site, T₀ represents a surface temperature measured by the thermal resistance sensor, q₁ is a value measured by the heat flux sensor, k is a tissue thermal conductivity, λ is a tissue density, ω is the blood perfusion, k₁ is a calibration coefficient for thermal resistance impact, and R is impact of contact thermal resistance.

According to a third aspect, the present disclosure provides a computer device, including a memory, a processor, and a computer program stored in the memory and executable on the processor, where the processor executes the computer program to perform the steps of the method described in the second aspect.

According to specific examples provided in this application, this application discloses the following technical effects:

The present disclosure emphatically proposes a modular user health status monitoring device, method, and system. The device includes a plurality of functional components, a sensing detection module, and a main control module. The plurality of functional components are detachably connected end to end to form a wristband or ring. The sensing detection module and the main control module are each located in any one of the functional components. The modules can be easily detached and combined, enabling convenient implementation of different measurement functions.

Furthermore, the device also includes a power module located in any one of the functional components. Existing wearable devices have limited battery capacity, and generally the entire device needs to be removed for charging. The modular ring-shaped monitoring device designed in the present disclosure allows direct replacement of the power module without needing to remove the entire device for charging, thereby improving monitoring continuity. Moreover, connections between the modules are elastic, meaning that there is a certain degree of elasticity between the modules, allowing for three-dimensional extension in the up-down and left-right directions, adapting to different sizes, making it more convenient to wear, and achieving monitoring that is comfortable, highly customizable, and accurate.

### BRIEF DESCRIPTION OF THE DRAWINGS

To describe the technical solutions in the examples of this application or in the prior art more clearly, the following briefly describes the accompanying drawings required for the examples. Apparently, the accompanying drawings in the following description show merely some examples of this application, and a person of ordinary skill in the art may still derive other accompanying drawings from these accompanying drawings without creative efforts.

FIG. 1 is a schematic structural diagram of a modular user health status monitoring device according to one or more embodiments of the present disclosure; and

FIG. 2 is a front view of the modular user health status monitoring device according to one or more embodiments of the present disclosure.

### DETAILED DESCRIPTION OF THE EMBODIMENTS

The technical solutions in the embodiments of the present disclosure are clearly and completely described below with reference to the drawings in the embodiments of the present disclosure. Apparently, the described embodiments are only some rather than all of the embodiments of the present disclosure. All other embodiments obtained by a person of ordinary skill in the art based on the embodiments of the present disclosure without creative efforts shall fall within the protection scope of the present disclosure.

An objective of the present disclosure is to provide a modular user health status monitoring device, method, and system, where each module can be easily detached and arbitrarily combined, enabling convenient implementation of different measurement functions.

To make the above objectives, features, and advantages of the present disclosure more obvious and easy to understand, the present disclosure will be further described in detail with reference to the accompanying drawings and specific implementations.

### Embodiment 1

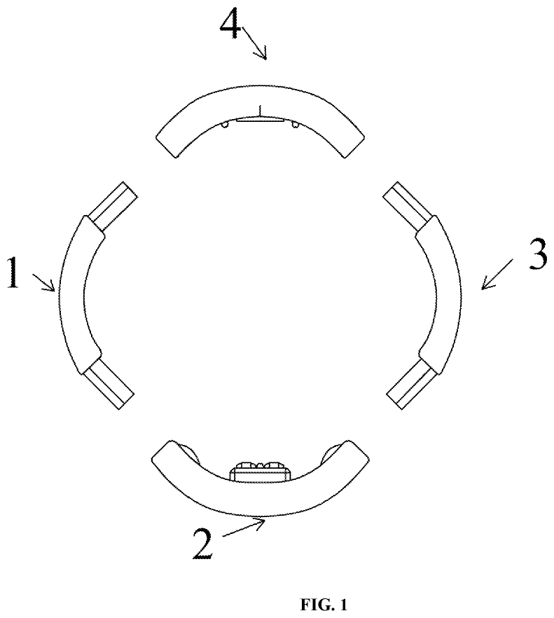

FIG. 1 is a schematic structural diagram of a modular user health status monitoring device according to one or more embodiments of the present disclosure. The monitoring device in the present disclosure includes: a plurality of functional components, a sensing detection module, a main control module, a storage module, a power module, a display module, and a data communication module. The sensing detection module, the storage module, the power module, the display module, and the data communication module are all connected to the main control module.

The plurality of functional components are detachably connected end to end to form a wristband or a ring. Referring to FIG. 1, four functional components are shown in this embodiment, namely, a first functional component 1, a second functional component 2, a third functional component 3, and a fourth functional component 4. The number of functional components is not limited in the present disclosure. In practical application, a person skilled in the art can adjust the number according to actual needs.

Specifically, the functional components are connected elastically, thereby enabling the wristband or ring to have a certain degree of elasticity, facilitating wearability. For example, an internal elastic component can be a spring, which allows for wearing on fingers/wrists of different sizes.

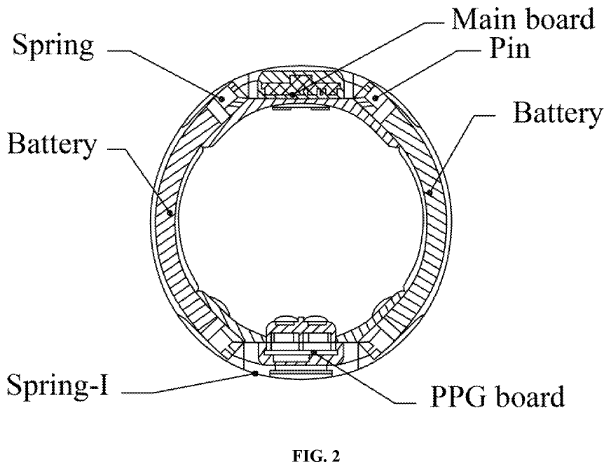

Referring to FIG. 2, the modules are fixedly connected by detachable pins. The pins provide a deformable and stretchable space with a certain length, enabling the ring structure to extend in multiple directions and thus increase its perimeter. The second functional component and the fourth functional component each have springs installed internally on both sides. A deformation length of the spring is slightly less than a stretchable deformation length of the pin and provides a force opposite to a deformation direction of the ring structure, so that the ring structure maintains good contact with fingers/wrists of different sizes without falling off. Conductive contacts/tracks are arranged within a deformable space of each functional component to provide electrical connections for the ring modules, including a power connection, and a signal connection between the sensing detection module and a main board.

Alternatively, the functional components may be fixed by a locking structure. The locking structure provides a deformable and stretchable space with a certain length, enabling the ring structure to extend in multiple directions and thus increase its perimeter. Springs are arranged inside overlapping areas of the functional components and provide a force that prevents the perimeter of the ring structure from increasing, so that the ring structure maintains good contact with fingers/wrists of different sizes without falling off. A flexible signal connection device, such as an electronic wire, is arranged within the deformable space of each functional component to provide electrical connections for the ring modules, including a power connection, and a signal connection between the sensing detection module and the main board.

The power module is disposed in the first functional component 1 and the third functional component 3, that is, the modular user health status monitoring device in this embodiment includes two power sources, which are detachable.

The sensing detection module and the main control module are disposed in the second functional component 2.

The storage module, the display module, and the data communication module are disposed in the fourth functional component 4.

The foregoing arrangement of the modules is merely an exemplary implementation in this embodiment, and the positions of the modules can be adjusted according to practical applications in the future.

Specifically, the sensing detection module includes: a MEMS gyroscope, an acceleration sensor, a heat flux sensor, a thermal resistance sensor, an electrochemical/fluorescence reaction sensor, a high-frequency electrical excitation electrode, a weak electrical detection electrode, an ink printing sensor, a photoelectric sensor, and a laser sensor.

### Embodiment 2

Corresponding to the modular user health status monitoring device provided in Embodiment 1, another embodiment of the present disclosure provides a modular user health status monitoring method, including the following steps:

Step 1: Acquire a PPG signal.

Specifically, the PPG signal is acquired by a photoelectric sensor, with a sampling frequency not less than 100 Hz and an analysis time window of 10 seconds.

Step 2: Calculate blood perfusion based on the PPG signal by using a decision algorithm.

Specifically, peripheral capillary oxygen saturation (Spo2) of a to-be-measured site is calculated from a multi-wavelength PPG signal based on the diffuse reflection principle.

Step 3: Acquire a surface temperature of a local tissue.

Based on Step 2, the surface temperature of the local tissue is measured by a thermal resistance sensor.

Step 4: Calculate a core temperature of a to-be-measured site based on the blood perfusion and the surface temperature of the local tissue.

The following formula is adopted:

$${T}_{c} = {T}_{0} + \frac{{q}_{1}}{k \sqrt{λ * ω}} + {k}_{1} R \qquad ( 1 )$$

where T_{c} represents the core temperature of the to-be-measured site, T₀ represents a surface temperature measured by the thermal resistance sensor, q₁ is a value measured by the heat flux sensor, k is a tissue thermal conductivity, λ is a tissue density, ω is the blood perfusion, k₁ is a calibration coefficient for thermal resistance impact, and R is impact of contact thermal resistance.

During the outward transfer of core temperature, the heat transfer analysis mainly involves three parts: heat transfer analysis within the organism, heat conduction condition at an interface between the organism and the sensor, and acquisition of a heat flow flowing into the sensor.

For the heat transfer analysis within the organism, the most widely used biological heat transfer equation at present is the Pennes equation (referring to formula 2), which considers heat conduction and metabolic heat, and simplifies the energy carried in the blood as an additional heat source term. The Pennes equation is simplified as follows:

$${ρ}_{t} {C}_{t} \frac{\partial T}{\partial t} = ∇ \cdot ( k ∇ T ) + {ω}_{b} {ρ}_{b} {C}_{b} ( {T}_{a} - T ) \qquad ( 2 )$$

where ρₜ is a tissue density, C_{b} is a tissue specific heat capacity, T is a tissue temperature, t is time, k is a tissue thermal conductivity, ρ_{b} is a blood density, Cₜ is a blood specific heat capacity, ω_{b} is a blood perfusion rate, and Tₐ is a blood temperature. This equation considers heat conduction and heat storage in living tissues and simulates biological tissues using a solid medium with density, heat capacity, and thermal conductivity. By continuously improving and refining this model, the temperature distribution pattern within the organism can be better understood, providing more effective tools and methods for biomedical research and applications.

Assuming the tissue extent is semi-infinite, and the temperature at the far boundary is the core temperature T_{c} and is stable, the boundary condition for the deep tissue is given as:

$$\frac{\partial T}{\partial x} {\vert}_{x \to ∞} = 0 \qquad ( 3 )$$

At a contact position of the interface between the organism and the sensor (x=0), factors such as dry skin and deep wrinkles may cause air gaps. Therefore, the heat flux needs to match the thermal contact resistance. This, together with the surface temperature (T₀) measured by the sensor, gives the second boundary condition:

$$- k \frac{\partial T}{\partial x} {\vert}_{x = 0} = \frac{1}{R} ( {T}_{0} - {T}_{skin} ) \qquad ( 4 )$$

R is a contact thermal resistance at the interface. Assuming there is no loss of heat flow into the sensor during contact, a heat flow q₀ at the interface and a temperature T₀ within the sensor can be obtained from the sensor.

From the above analysis, the simplified Pennes equation and the boundary conditions can be combined as follows:

$$\{ \begin{matrix}0 = α \frac{{\partial }^{2} T}{\partial {x}^{2}} - {ω}_{b} ( T - {T}_{c} ) \\ - k \frac{\partial T}{\partial x} {\vert}_{x = 0} = \frac{1}{{R}^{′}} ( {T}_{0} - {T}_{skin} ) \\ \frac{\partial T}{\partial x} {\vert}_{x \to ∞} = 0\end{matrix} \qquad ( 5 )$$

where ω_{b}, ρ_{b}, C_{b} are the perfusion rate, density, and specific heat capacity of blood within the tissue, respectively; k is the tissue thermal conductivity; an expression for α is

$$\frac{k}{{ρ}_{b} {C}_{b}} ,$$

and the subscripts skin and c represent the skin and core, respectively.

By solving formula (5), the tissue temperature distribution under steady-state conditions can be obtained:

$$T = {T}_{c} + ( {T}_{0} - {T}_{c} ) [ \frac{1}{1 + R k \sqrt{{ω}_{b} / α}} ] {e}^{- \sqrt{{ω}_{b} / α} x} \qquad ( 6 )$$

The corresponding heat flow at the skin is:

$$q ( x = 0 ) = - k ( {T}_{0} - {T}_{c} ) [ \frac{\sqrt{{ω}_{b} / α}}{1 + R k \sqrt{{ω}_{b} / α}} ] \qquad ( 7 )$$

This value can be obtained by the sensor. From the above equation, the relationship between the core temperature T_{c} and the contact thermal resistance R as well as the sensor temperature T₀ can be determined:

$${T}_{c} = {T}_{0} + \frac{{q}_{1}}{k \sqrt{λ * {ω}_{b}}} + {k}_{1} R \qquad ( 8 )$$

T_{c} is the core temperature to be measured; T₀ is the surface temperature measured by the thermal resistance sensor; q₁ is the value measured by the heat flux sensor; k is the tissue thermal conductivity; [symbol] is a constant related to the tissue density and specific heat capacity; ω_{b} is the blood perfusion; k₁ is the calibration coefficient for thermal resistance impact; and R is the impact of contact thermal resistance. It can be seen that the second term of the formula is related to blood perfusion, which can reflect the thermoregulatory effect inside human tissues, and the third term is related to contact thermal resistance, which can express the impact of the contact interface on heat transfer.

Step 5: Calculate blood glucose of a user, specifically including:
S5.1: Collect blood glucose monitoring data, where the blood glucose monitoring data includes: timestamps and corresponding blood glucose, as well as multi-wavelength PPG original signals, core temperature T_{c} of the to-be-measured site, and Spo2 of the to-be-measured site.
S5.2: Preprocess the blood glucose monitoring data.

First, missing values, outliers, and noise are processed; second, useful features are extracted from the original data, and in addition to the aforementioned training set data, time, diet, exercise, medication use, and other data are fused; finally, new features are created, such as the trend and rate of change of blood glucose.
S5.3: Construct a plurality of deep learning sub-models.
S5.4: Train the plurality of deep learning sub-models based on the preprocessed blood glucose monitoring data.

The deep learning sub-models can adopt Long Short-Term Memory (LSTM), Generative Adversarial Network (GAN), Bidirectional Gated Recurrent Unit (Bi-GRU), and attention mechanisms, and the like. The sub-models are trained using the training dataset to optimize model parameters, perform cross-validation, and adjust hyperparameters, thereby avoiding overfitting.

S5.5: Perform weighted fusion on the plurality of trained deep learning sub-models using a model fusion algorithm to obtain a final blood glucose prediction model.

Specifically, model fusion techniques, such as weighted averaging and voting, are used to fuse the prediction results of different models to improve overall performance.

S5.6: Predict the blood glucose of the user based on the blood glucose prediction model.

New blood glucose monitoring data are input to the trained blood glucose prediction model for prediction. The trained blood glucose prediction model outputs predicted blood glucose and generates corresponding prompt information based on thresholds, such as hypoglycemia, normal, and hyperglycemia.

### Embodiment 3

A computer system is provided, including a memory, a processor, and a computer program stored in the memory and executable on the processor, where the computer program is executed by the processor to perform the steps of the modular user health status monitoring method in Embodiment 2.

Those of ordinary skill in the art may understand that all or some of the procedures in the method of the foregoing embodiments may be implemented by a computer program instructing related hardware. The computer program may be stored in a nonvolatile computer-readable storage medium. When the computer program is executed, the procedures in the embodiments of the foregoing method may be performed. Any reference to a memory, a database, or other media used in the embodiments of the present disclosure may include a non-volatile and/or volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM) or an external cache memory. As an illustration rather than a limitation, the RAM may be in various forms, such as a static random access memory (SRAM) or a dynamic random access memory (DRAM). The database in the embodiments of the present disclosure may include at least one of a relational database and a non-relational database. The non-relational database may include a distributed database based on a blockchain, but is not limited thereto. The processor in the embodiments of the present disclosure may be a general processor, a central processor, a graphics processor, a digital signal processor (DSP), a programmable logic device, and a data processing logic device based on quantum computing, but is not limited thereto.

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of this application. The description of the foregoing examples is used to help illustrate the method of this application and the core principles thereof. In addition, those of ordinary skill in the art can make various modifications in terms of specific implementations and scope of application in accordance with the teachings of this application. In conclusion, the content of the present specification shall not be construed as a limitation to the present disclosure.

What is claimed is:

1. A modular user health status monitoring device, comprising:
a plurality of functional components, wherein the plurality of functional components are detachably connected end to end to form a wristband or a ring;
a sensing detection module, located in any one of the functional components; and
a main control module, located in any one of the functional components and connected to the sensing detection module.

2. The modular user health status monitoring device according to claim 1, further comprising: a storage module, located in any one of the functional components and connected to the main control module.

3. The modular user health status monitoring device according to claim 1, further comprising: a power module, located in any one of the functional components and connected to the sensing detection module and the main control module.

4. The modular user health status monitoring device according to claim 1, further comprising: a display module, located in any one of the functional components and connected to the main control module.

5. The modular user health status monitoring device according to claim 1, further comprising: a data communication module, located in any one of the functional components and connected to the main control module.

6. The modular user health status monitoring device according to claim 1, wherein the sensing detection module comprises: a Micro-Electro-Mechanical System (MEMS) gyroscope, an acceleration sensor, a heat flux sensor, a thermal resistance sensor, an electrochemical/fluorescence reaction sensor, a high-frequency electrical excitation electrode, a weak electrical detection electrode, an ink printing sensor, a photoelectric sensor, and a laser sensor.

7. A modular user health status monitoring method, wherein the monitoring method is applied to the device according to claim 1, and the monitoring method comprises:
acquiring a photoplethysmography (PPG) signal;
calculating blood perfusion based on the PPG signal by using a decision algorithm;
acquiring a surface temperature of a local tissue; and
calculating a core temperature of a to-be-measured site based on the blood perfusion and the surface temperature of the local tissue.

8. The modular user health status monitoring method according to claim 7, further comprising: calculating blood glucose of a user, comprising:
collecting blood glucose monitoring data, wherein the blood glucose monitoring data comprises: timestamps and corresponding blood glucose, as well as multi-wavelength PPG original signals, a core temperature T_{c} of the to-be-measured site, and peripheral capillary oxygen saturation (Spo2) of the to-be-measured site;
preprocessing the blood glucose monitoring data;
constructing a plurality of deep learning sub-models;
training the plurality of deep learning sub-models based on the preprocessed blood glucose monitoring data;
performing weighted fusion on the plurality of trained deep learning sub-models using a model fusion algorithm to obtain a final blood glucose prediction model; and
predicting the blood glucose of the user based on the blood glucose prediction model.

9. The modular user health status monitoring method according to claim 7, wherein a formula for calculating the core temperature of the to-be-measured site based on the blood perfusion and the surface temperature of the local tissue is as follows:
$${T}_{c} = {T}_{0} + \frac{{q}_{1}}{k \sqrt{λ * ω}} + {k}_{1} R ;$$
wherein T_{c} represents the core temperature of the to-be-measured site, T₀ represents a surface temperature measured by the thermal resistance sensor, q₁ is a value measured by the heat flux sensor, k is a tissue thermal conductivity, λ is a tissue density, ω is the blood perfusion, k₁ is a calibration coefficient for thermal resistance impact, and R is impact of contact thermal resistance.

10. A computer system, comprising a memory, a processor, and a computer program stored in the memory and executable on the processor, wherein the computer program, when executed by the processor, causes the processor to perform the steps of the method according to claim 7.

11. The modular user health status monitoring method according to claim 7, further comprising: a storage module, located in any one of the functional components and connected to the main control module.

12. The modular user health status monitoring method according to claim 7, further comprising: a power module, located in any one of the functional components and connected to the sensing detection module and the main control module.

13. The modular user health status monitoring method according to claim 7, further comprising: a display module, located in any one of the functional components and connected to the main control module.

14. The modular user health status monitoring method according to claim 7, further comprising: a data communication module, located in any one of the functional components and connected to the main control module.

15. The modular user health status monitoring method according to claim 7, wherein the sensing detection module comprises: a Micro-Electro-Mechanical System (MEMS) gyroscope, an acceleration sensor, a heat flux sensor, a thermal resistance sensor, an electrochemical/fluorescence reaction sensor, a high-frequency electrical excitation electrode, a weak electrical detection electrode, an ink printing sensor, a photoelectric sensor, and a laser sensor.

16. The computer system according to claim 10, further comprising: calculating blood glucose of a user, comprising:
collecting blood glucose monitoring data, wherein the blood glucose monitoring data comprises: timestamps and corresponding blood glucose, as well as multi-wavelength PPG original signals, a core temperature T_{c} of the to-be-measured site, and peripheral capillary oxygen saturation (Spo2) of the to-be-measured site;
preprocessing the blood glucose monitoring data;
constructing a plurality of deep learning sub-models;
training the plurality of deep learning sub-models based on the preprocessed blood glucose monitoring data;
performing weighted fusion on the plurality of trained deep learning sub-models using a model fusion algorithm to obtain a final blood glucose prediction model; and
predicting the blood glucose of the user based on the blood glucose prediction model.

17. The computer system according to claim 16, wherein a formula for calculating the core temperature of the to-be-measured site based on the blood perfusion and the surface temperature of the local tissue is as follows:
$${T}_{c} = {T}_{0} + \frac{{q}_{1}}{k \sqrt{λ * ω}} + {k}_{1} R ;$$
wherein T_{c} represents the core temperature of the to-be-measured site, T₀ represents a surface temperature measured by the thermal resistance sensor, q₁ is a value measured by the heat flux sensor, k is a tissue thermal conductivity, λ is a tissue density, ω is the blood perfusion, k₁ is a calibration coefficient for thermal resistance impact, and R is impact of contact thermal resistance.
